(19) United States
(12) Patent Application Publication
(10) Pub. No.: US 2009/0209604 A1
(43) Pub. Date: Aug. 20, 2009

(54) Topical combination therapy for treating acne

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 12/069,994

(22) Filed: Feb. 14, 2008

(51) Int. Cl. A61K 31/4164; A61P 17/10

(52) U.S. Cl. 514/390

(57) ABSTRACT

Described are aqueous topical compositions containing a combination of benzoyl peroxide, at least one dicarboxylic acid having 6 to 12 carbon atoms, and at least one vitamin B.sub.3 compound in aqueous vehicles containing at least one 1,2-alkanediol having from 5 to 7 carbon atoms. The compositions are suitable for treatment of acne. Also described are methods for topically delivering a medicament to a human patient, in need of the medicament by topically administering to the patient the compositions of the present invention.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to topical compositions for treatment of acne. In particular, the invention relates to topical compositions containing a combination of benzoyl peroxide, at least one dicarboxylic acid having 6 to 12 carbon atoms, and at least one vitamin B.sub.3 compound in aqueous vehicles containing at least one 1,2-alkanediol having 5 to 7 carbon atoms.

### BACKGROUND OF THE INVENTION

Benzoyl peroxide is one of most frequently used acne treatment medications. Common side effects are irritation, skin drying, flaking, and burning, particularly at higher concentrations, generally at 5% or greater.

Dicarboxylic acids having from 6 to 12 carbon atoms have been known as an effective treatment for acne and rosacea. In particular, azelaic acid (having 9 carbon atoms) is an active ingredient in a number of prescription products for treatment of acne and rosacea. For example, AZELEX.™ cream (Allergan Corp. Irvine, Calif.) containing 20% of azelaic acid is used to treat acne. FINACEA.™ gel (INTENDIS, Pine Brook, N.J.) containing 15% of azelaic acid is used to treat rosacea. Due to acidic nature of azelaic acid, stinging and skin irritation are common side effects at such concentrations.

Niacinamide has also been used for treatment of acne. For example, NICOMIDE-T.™ gel or cream (DUSA Pharmaceuticals, Wilmington, Mass.) containing 4% of niacinamide is used to treat acne. However, its therapeutic efficacy is generally moderate.

For the treatment of many dermatological disorders including acne, it is often preferable to use a combination therapy containing a combination of active ingredients for enhanced therapeutic efficacy and reduced side effects.

Therefore, there is a need for a safe and effective topical treatment of acne without disadvantages of the prior art.

There is also a need for an enhanced therapy for acne by employing a topical composition containing a combination of benzoyl peroxide, at least one dicarboxylic acid having from 6 to 12 carbon atoms, and at least one vitamin B.sub.3 compound at a therapeutically safe and effective level.

### SUMMARY OF THE INVENTION

Accordingly, it is an object of the invention to provide a method for treating acne by topically administering to a human patient in need of such treatment a safe and therapeutically effective amount of a composition containing benzoyl peroxide, at least one dicarboxylic acid having from 6 to 12 carbon atoms, and at least one vitamin B.sub.3 compound in an aqueous vehicle containing at least one 1,2-alkanediol having from 5 to 7 carbon atoms.

Another object of the invention is to formulate a topical composition containing benzoyl peroxide, at least one dicarboxylic acid having from 6 to 12 carbon atoms, and at least one vitamin B.sub.3 compound in an aqueous vehicle containing at least one 1,2-alkanediol having from 5 to 7 carbon atoms.

A further object of the invention is to formulate a topical composition for the combination therapeutic treatment of acne.

Still other objects and advantages of the invention will, in part, be obvious and will, in part, be apparent from the following detailed description of the preferred embodiments.

### BRIEF DESCRIPTIONS OF THE DRAWINGS

FIG. 1 shows chemical structure of a dicarboxylic acid.

FIG. 2 shows chemical structure of 1,2-pentanediol, 1,2-hexanediol, and 1,2-heptanediol.

### DETAILED DESCRIPTION OF THE PREFERRED EMBODIMENTS

In the described embodiments, disclosed are topical compositions containing benzoyl peroxide, at least one dicarboxylic acid having from 6 to 12 carbon atoms, and at least one vitamin B.sub.3 compound in an aqueous vehicle containing at least one 1,2-alkanediol having from 5 to 7 carbon atoms. Also disclosed are methods for treating acne.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘pharmaceutically acceptable’, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term ‘safe and effective’, as used herein, means a concentration of an active ingredient or an amount of a composition, that is sufficient enough to significantly and positively modify the condition to be treated but low enough to avoid serious side effects, within the scope of sound medical advice.

All percentages referred to in this specification are percentages by weight of the total composition unless otherwise indicated.

Benzoyl peroxide is commercially available from various vendors, for example, Aldrich Chemical Company (Milwaukee, Wis.).

Concentration of benzoyl peroxide in the composition of the present invention may be in the range of from about 1% to about 15%, preferably from about 2% to about 10%.

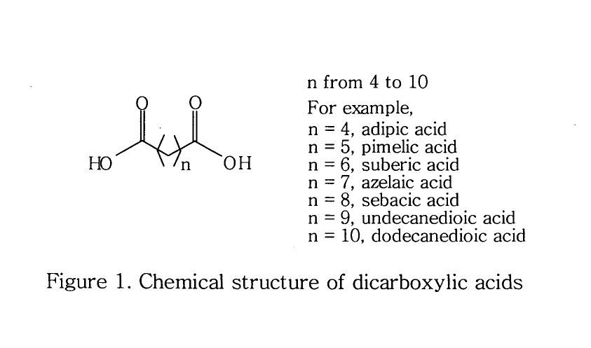

Chemical structure of a dicarboxylic acid in accordance with the present invention is shown in FIG. 1. The alkyl chain in between the two terminal carboxylic groups is a saturated straight alkyl group. Examples of the suitable dicarboxylic acid are adipic acid, pimelic acid, suberic acid, azelaic acid, sebacic acid, undecanedioic acid, dodecanedioic acid, and any suitable combinations thereof. These dicarboxylic acids are commercially available from various vendors, for example, Aldrich Chemical Company (Milwaukee, Wis.). Azelaic acid is preferred dicarboxylic acid.

Concentration of the dicarboxylic acid in the composition of the present invention may be in the range of from about 1% to about 30%, preferably from about 2% to about 25%, more preferably from about 5% to about 20%.

Vitamin B.sub.3 family of compounds includes niacinamide (also known as nicotinamide), isonicotinamide, niacin (also known as nicotinic acid), and isonicotinic acid. Isonicotinamide is a structural isomer of nicotinamide. Isonicotinic acid is a structural isomer of nicotinic acid.

Examples of the vitamin B.sub.3 compounds in the composition of the present invention are niacinamide, isonicotinamide, niacin, isonicotinic acid and suitable combinations thereof. Examples of the suitable combinations include niacinamide and niacin, niacinamide and isonicotinic acid, niacinamide and isonicotinamide and niacin, and so on.

Concentration of niacinamide or isonicotinamide in the composition of the present invention may be in the range of from about 1% to about 10%, preferably from about 2% to about 5%.

Concentration of niacin or isonicotinic acid in the composition of the present invention may be in the range of from about 0.01% to about 2%, preferably from about 0.05% to about 0.5%.

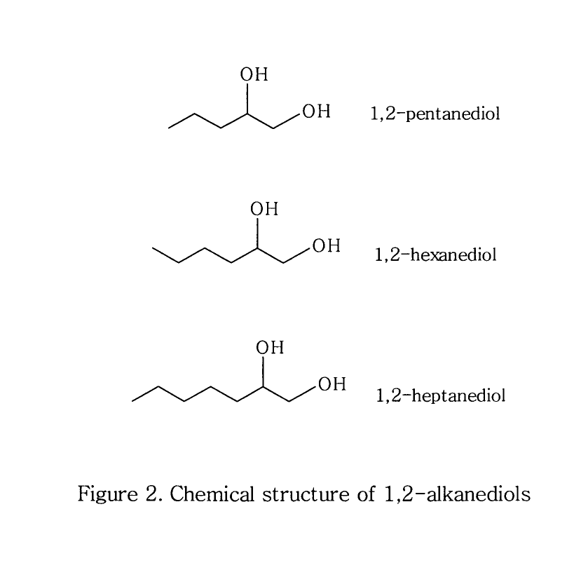

Chemical structure of 1,2-alkanediols having from 5 to 7 carbon atoms in accordance with the present invention is shown in FIG. 2. The alkyl chain in the 1,2-alkanediols is a saturated straight alkyl group having from 5 to 7 carbon atoms. The 1,2-alkanediols in accordance with the present invention are 1,2-pentanediol, 1,2-hexanediol, 1,2-heptanediol, and any suitable combinations thereof. These 1,2-alkanediols are commercially available, for example, from Aldrich Chemical Company (Milwaukee, Wis.). They may also be chemically synthesized, for example, by catalytic hydroxylation of the corresponding terminal alkenes (also known as 1-alkenes) using a combination of hydrogen peroxide and osmium tetroxide. 1,2-Hexanediol is preferred.

Concentration of the 1,2-alkanediol in the composition of the present invention may be in the range of from about 5% to about 70%, preferably from about 10% to about 60%, more preferably from about 15% to about 50%.

The composition, in accordance with the present invention, may be in the form of a gel, lotion, cream, or foam. In one embodiment, the composition is a gel. Therefore, the aqueous composition preferably contains a gelling agent. Any gelling agent that is dispersible in the carrier, physically and chemically compatible with the ingredients in the composition, and forms a gel of substantially uniform consistency is suitable for use in the present invention.

Examples of the suitable gelling agents are inorganic based gelling agents, polycarbohydrate based gelling agents, and polyacrylic acid based gelling agents. Examples of the suitable inorganic gelling agents are silica gel and clay. Examples of the suitable polycarbohydrate based gelling agents are hydroxyethylcellulose, hydroxypropylcellulose, and xanthan gum. Examples of the suitable polyacrylic acid based gelling agents are CARBOPOL Brand 934, 940, 941, 980, Ultrez 10, and Ultrez 20 (available from Noveon Corp., Cleveland, Ohio). Combinations of the above types of gelling agents are also suitable as the gelling agents.

The composition, in accordance with the present invention, may optionally contain nonionic surfactants. Nonionic surfactants are generally compounds formed by the condensation of alkylene oxide groups such as, for example, ethylene oxide and propylene oxide, with a lipophilic compound. Examples of classes of nonionic surfactants are:

(a). Polysorbates, or sorbitol or sucrose esters of fatty acids. Examples of the suitable polysorbates are polysorbate-20, polysorbate-40, polysorbate-60, polysorbate-65, polysorbate-80, and polysorbate-85. Examples of the suitable sorbitol esters of fatty acids are sorbitan monooleate, sorbitan monopalmitate, sorbitan monolaurate, sorbitan monostearate, sorbitan monoisostearate, sorbitan sesquioleate, sorbitan trioleate, sorbitan tristearate, sorbitan monooleate ethoxylate EO 20 mole, sorbitan monolaurate ethoxylate EO 20 mole, sorbitan monopalmitate ethoxylate EO 20 mole, sorbitan monostearate ethoxylate EO 20 mole, and so on.

(b). Alkyl polysaccharides having a lipophilic group of 6 to 30 carbon atoms and polysaccharide group such as glucose, galactose, and so on. Examples of the suitable alkyl polysaccharides are octyl, nonydecyl, undecyl, dodecyl, hexadecyl, octadecyl gluocosides, galactosides, and so on.

(c). Polyol or polyethylene glycol (PEG) esters of fatty acids and PEG ethers of fatty alcohols. Examples of the suitable fatty esters or ethers are glyceryl stearate, glyceryl distearate, PEG-40 stearate, PEG-50 stearate, PEG-100 stearate, oleth-5, oleth-10, oleth-20, laureth-23, ceteareth-20, ceteareth-21, steareth-10, steareth-21, and so on.

The composition may contain anti-inflammatory agents. Examples of the suitable anti-inflammatory agents are salicylic acid and pharmaceutically acceptable salts or esters thereof, ketoprofen, ibuprofen, cat's claw extract, green tea extract, curcumin extract, and dandelion extract.

The composition, in accordance with the present invention, may contain conventional amounts of customary auxiliaries and additives provided that such auxiliaries and additives are physically and chemically compatible with the ingredients in the composition and do not reduce the therapeutic efficacy of the composition.

Furthermore, the auxiliaries and additives may not impair favorable cosmetic properties of the composition of the present invention.

Examples of customary auxiliaries and additives include, but not limited to: moisturizing compounds, vitamins, antioxidants, film-forming polymers, and other desirable ingredients.

The composition of the present invention may contain moisturizing compounds including, but not limited to: allantoin, urea, N-2-hydroxyethyl urea, additional polyhydric alcohols (also known as polyols) other than 1,2-alkanediols having from 5 to 7 carbon atoms, polyol ethers and esters, low molecular weight polyethylene glycols, lactates, sugars, methyl glucose ethers, sodium pyrrolidone carboxylic acid, sodium hyaluronate, panthenol, and hyaluronic acid, triglycerides, fatty acids and esters, fatty alcohols, or combinations of the suitable moisturizing compounds.

Examples of the suitable polyols are glycerin (also known as glycerol), propylene glycol (also known as 1,2-propanediol), 1,3-propanediol, 1,2-butanediol, 1,3-butanediol, 2,3-butanediol, 1,5-pentanediol, 2-methyl-2,4,-pentanediol (also known as hexylene glycol), 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, and hexylene glycol. Examples of the suitable low molecular weight polyethylene glycols (PEG) are PEG-4, PEG-6, PEG-8, and PEG-10. Examples of the suitable lactates are ammonium lactate, sodium lactate, and potassium lactate. Examples of the suitable methyl glucose ethers are methyl gluceth-10 and methyl gluceth-20.

Examples of the suitable vitamins are tocopherol (vitamin E) and derivatives thereof, vitamin B.sub.2 and derivatives thereof, vitamin C and derivatives thereof, and combinations of the suitable vitamins thereof.

Examples of the suitable antioxidants are ascorbic acid, sodium sulfite, green tea extract, grape seed extract, flavonoids, carnosine and derivatives thereof, glutathione, cysteine and derivatives thereof, proline, carnitine and derivatives thereof, and combinations of the suitable antioxidants thereof.

The film forming properties of polymers may help maintain active ingredients on the site of application, which in turn may help enhance therapeutic efficacy of the composition. Examples of the suitable film forming polymers are hydroxypropyl cellulose, polyvinylpyrrolidone, polyvinylpyrrolidone vinylacetate copolymer, polyvinyl alcohol, and combinations thereof.

The composition of the present invention may also contain one or more other desirable ingredients including, but not limited to: skin penetration enhancers, herbal extracts, chelating agents, preservatives, colorants, fragrances, and so on. Examples of the suitable herbal extracts are garlic extract, grape seed extract, and onion extract. Examples of the suitable skin penetration enhancers are propylene glycol, dimethyl isosorbide, and N-methyl-1-pyrrolidone. Examples of the suitable chelating agents are EDTA (ethylenediaminetetraacetic acid), EGTA [ethylenebis(oxyethylenenitrilo)tetraacetic acid], and pharmaceutically acceptable salts thereof.

The composition of the present invention may be used for topical treatment of dermatological disorders that are responsive to the combination therapy. In accordance with the method of the present invention, the composition containing a combination of benzoyl peroxide, dicarboxylic acid having 6 to 12 carbon atoms, at least one vitamin B.sub.3 compound in an aqueous vehicle is topically applied in a therapeutically safe and effective amount to skin surfaces of human beings in need of such therapy.

The therapeutic method of the present invention may be used to treat any disorder that is responsive, or potentially responsive, to the combination therapy. Examples of the disorders that are suitable to be treated include bacterial infections, and certain infectious diseases that may be treated topically.

Preferably, the disorder to be treated is acne.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

This example is to prepare a topical composition containing 3% of benzoyl peroxide, 10% of azelaic acid, and 4% of niacinamide in an vehicle containing 25% of 1,2-hexanediol using hydroxypropylcellulose as the gelling agent. The aqueous composition in accordance with the invention was prepared as follows:

| Component | Amount (weight percentage) |
|---|---|
| Benzoyl peroxide | 3% |
| Azelaic acid | 10% |
| Niacinamide | 4% |
| 1,2-Hexanediol | 25% |
| Polysorbate 60 | 1.5% |
| Allantoin | 0.5% |
| Hydroxypropylcellulose | 1.5% |
| Water | q.s |

Azelaic acid was added to 1,2-hexandiol and the mixture was kept at about 55.degree. C., while stirring until azelaic acid was solubilized. Niacinamide, polysorbate 60, and allantoin were added to water (q.s. to 100) and allowed to be solubilized. The gelling agent, hydroxypropylcellulose, (Kiucel M F, Hercules, Inc. Wilmington, Del.), was added to the water solution. The mixture was maintained at room temperature, while stirring until gelled. Micronized benzoyl peroxide was shifted into the gel, while mixing until a uniform opaque gel was formed. The azelaic acid solution was then added to the gel, while mixing until a uniform gel product was formed.

### EXAMPLE 2

This example is to prepare a topical composition containing 3% of benzoyl peroxide, 10% of azelaic acid, and 4% of niacinamide in an vehicle containing 25% of 1,2-hexanediol using a CARBOPOL as the gelling agent. The aqueous composition in accordance with the invention was prepared as follows:

| Component | Amount (weight percentage) |
|---|---|
| Benzoyl peroxide | 3% |
| Azelaic acid | 10% |
| Niacinamide | 4% |
| 1,2-Hexanediol | 25% |
| Polysorbate 60 | 1.5% |
| Allantoin | 0.5% |
| CARBOPOL | 1.5% |
| Water | q.s |

Azelaic acid was added to 1,2-hexandiol and the mixture was kept at about 55.degree. C., while stirring until azelaic acid was solubilized. Niacinamide, polysorbate 60, and allantoin were added to water (q.s. to 100) and allowed to be solubilized. Micronized benzoyl peroxide was added to the solution. Then, the azelaic acid solution was added. The gelling agent, CARBOPOL 980, was added to the mixture in a shifted fashion, while stirring until gelled. The final product was a white cream.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A topical composition, comprising, by weight of the total composition:
benzoyl peroxide in an amount of from about 1% to about 15%;
at least one dicarboxylic acid having from 6 to 12 carbon atoms, in an amount from about 1% to about 30%;
a vitamin B.sub.3 compound selected from the group consisting of niacinamide, isonicotinamide, niacin, isonicotinic acid, and combinations thereof, in an amount from about 0.01% to about 10%;
a 1,2-alkanediol selected from the group consisting of 1,2-pentanediol, 1,2-hexanediol, 1,2-heptanediol, and combinations thereof, in an amount of from about 5% to about 70%;
a pharmaceutically acceptable carrier.

2. The composition of claim 1 wherein the benzoyl peroxide is in an amount of from about 2% to about 10%.

3. The composition of claim 1 wherein the dicarboxylic acid is azelaic acid.

4. The composition of claim 1 wherein the dicarboxylic acid is in an amount of from about 2% to about 20%.

5. The composition of claim 3 wherein the azelaic acid is in an amount of from about 2% to about 20%.

6. The composition of claim 1 wherein the 1,2-alkanediol is in an amount of from about 10% to about 60%.

7. The composition of claim 1 wherein the 1,2-alkanediol is 1,2-hexanediol.

8. The composition of claim 7 wherein the 1,2-hexanediol is in an amount of from about 10% to about 60%.

9. The composition of claim 1 wherein the vitamin B.sub.3 compound is niacinamide.

10. The composition of claim 9 wherein the niacinamide is in an amount of from about 2% to about 10%.

11. The composition of claim 1 wherein the vitamin B.sub.3 compound is a combination of niacinamide and niacin.

12. The composition of claim 11 wherein the niacinamide is in an amount of from about 1% to about 10% and the niacin is in an amount of from about 0.05% to about 0.5%.

13. The composition of claim 1 wherein the dicarboxylic acid is azelaic acid, the vitamin B.sub.3 compound is niacinamide, and the 1,2-alkanediol is 1,2-hexanediol.

14. A topical composition, comprising, by weight of the total composition:
benzoyl peroxide in an amount of from about 2% to about 10%;
azelaic acid in an amount from about 2% to about 30%;
niacinamide in an amount from about 1% to about 10%;
1,2-hexandiol in an amount of from about 5% to about 50%;
a pharmaceutically acceptable carrier.

15. The composition in claim 14 wherein the 1,2-hexanediol is in an amount of from about 10% to about 30%.

16. The composition in claim 14 wherein the azelaic acid is in an amount of from about 5% to about 20%.

17. The composition of claim 14 wherein the niacinamide is in an amount of from about 2% to about 5%.

18. A method for treating a dermatological disorder comprising administering a therapeutically safe and effective amount of the composition as claimed in claim 1 to a human patient in need of such treatment.

19. The method of claim 16 wherein the dermatological disorder is acne.
